(19) United States
(12) Patent Application Publication
(10) Pub. No.: US 2012/0263784 A1
(43) Pub. Date: Oct. 18, 2012

(54) EMERGENCY CONTRACEPTIVE

(75) Inventors: Ambikanandan Rajnarayan Misra, Vadadora (IN); Narendra Ishwarlal Gandhi, Mumbai (IN); Mannalal Ramgopal Bajaj, Mumbai (IN); Bharat Babulal Shah, Mumbai (IN); Rajan Shantaram Samant, Mumbai (IN)

(73) Assignee: LYKA LABS LIMITED, Mumbai, Maharashtra (IN)

(21) Appl. No.: 13/509,529

(22) PCT Filed: Oct. 12, 2010
(86) PCT No.: PCT/IN2010/000676

§ 371 Date: May 11, 2012

(30) Foreign Application Priority Data
Oct. 12, 2009 (IN) 2373/MUM/2009

(51) Int. Cl. A61K 31/57; A61K 31/565; A61P 15/18; A61K 9/127

(52) U.S. Cl. 424/450; 514/179; 514/170

(57) ABSTRACT

The present invention discloses an emergency contraceptive formulation for nasal and/or pulmonary administration comprising of levonorgestrel optionally in combination with ethylestradiol. The said formulation can be in form of solution, suspension or carrier based systems selected from microemulsion and liposomes.

### FIELD OF INVENTION

The current invention is in the field of drug delivery. More particularly the current invention relates to nasal and/or pulmonary route for administration of emergency contraceptive formulation comprising synthetic progesterone or synthetic estrogen or a combination of both.

### BACKGROUND OF INVENTION

Birth control, more commonly known as “contraception”, is a regimen of one or more actions, devices, sexual practices or medications followed in order to deliberately prevent or reduce likelihood of pregnancy or childbirth. Birth control involves about 3 routes namely, prevention of fertilization of ovum or “contraception”; prevention of implantation of blastocyst, or contragestion and chemical or surgical induction of abortion of embryo or foetus.

The history of birth control began with discovery of relationship between coitus and conception. Traditionally, coitus interrupts or withdrawal or pull-out method for voluntary prevention of entry or sperms or semen in female reproductive system, use of pessaries or use of abortifacient herbs.

Modern methods involve use of oral contraceptive pills. Oral contraceptives (OCs) allow effective and convenient family planning for women and couples worldwide, and have revolutionized the reproductive lives of millions of women since their introduction in the 1960s. Oral Contraceptives are now also used to avoid pregnancy in case of emergency contraception (EC) as a backup method for unprotected sexual intercourse.

Eventhough, oral contraceptives (OCs) allow effective and convenient family planning for women and couples worldwide, and have revolutionized the reproductive lives of millions of women since their introduction in the 1960s.

All oral contraceptive drugs interfere with the production and action of endogenously synthesized steroid hormones (Kent et al., 2002). Also, induction of hepatic enzymes by oral contraceptives may interference with potency and duration of other medications such as anticoagulants (Ellison et al., 2000), antibiotics, or anticonvulsant drugs. In addition, orally administered steroids interfere to different degrees with hepatic protein synthesis of pro-coagulatory and fibrinolytic proteins (Rosing et al., 1999) and fatty liver as a consequence of long-term treatment. It is also likely that factors originating from or due to hepatic metabolism of exogenous steroids play a role in hypertension and dyslipidaemia, side effects frequently observed with oral contraceptive treatment. Therefore there is a need of development of female contraceptives other than presently available oral contraception without compromise of safety and user compliance.

New materials and new technologies have stimulated pharmaceutical researchers to identify and use alternatives to the classical oral and injectable routes. One of the routes currently being studied is the nasal way. The intranasal administration provides a useful way of taking a range of systemic drugs. The compliance of patients who require long-term medication has been shown to be better due to the simplicity and ease of administration when compared to the parenteral route (Pontiroli et al., 1985; Hirai et al., 1981). Also, the rate of absorption, plasma concentration and pharmacokinetics following nasal route of administration is often compares well to that obtained by intravenous medication because of rich vasculature and high permeability of nasal mucosa. In selected drugs the pharmacokinetics relating to drug absorption and metabolism via the nose are more favorable (Chien et al, 1989; Wuthrich and Buri, 1987; Pontiroli et al., 1989).

It has recently been shown that the bioavailability of the steroidal drugs progesterone, β-oestradiol, 17α-ethinyloestradiol when given via nasal route in rats is greatly superior to that via the oral route (Bawarshi-Nassar et al., 1989). Also, study involving nasal administration of norethisterone shows superiority of this route (Anand Kumar et al., 1991). The bioavailability of Testosterone was found similar to that of i.v. route when given nasally (Hussain et al., 1984). The large number of fenestrated capillaries just below the surface epithelium may well contribute to absorption (Fisher, 1990).

Weakly cross-linked polyacrylates as carbomers by Ca²⁺ complexation are able to trigger the reversible opening of the tight-junction between the cells and to allow the paracellular transport of peptides (Luessen et al., 1996, Junginger and Verhoef, 1998). Chitosans have been shown to have similar properties to reversibly open the tight junctions. This mechanism is thought to occur by ionic charge transfer between the positive charge of chitosan molecule and the negative charges (sulfate and sialine groups) of the glycocalix (Thanou et al., 2000).

However, for the drugs of very short biological half-life, the rapid absorption is unfavourable to sustain the drug level in the systemic circulation, and the large mucociliary clearance of the nasal mucosa (Schipper et al., 1991) may cause poor absorption of certain drugs. The mucociliary clearance under normal conditions rapidly clears the applied material since there is a little time of contact between the drug and the mucosa. Therefore, employing nasal delivery for prolonged release required the development of particular strategies in order to keep the substance on the mucosa for a long time without altering the functionality of the nose. Prolonging the contact of the drug with the absorptive surfaces by means of an appropriate delivery system can increase the bioavailability of intranasally administered drug. It was reported that bioavailability of drug with the bioadhesive compound such as carbopol and chitosan was increased by durability of the drug concentration in plasma by bioadhesivity of polymer to the mucus.

Weakly cross-linked polyacrylates as carbomers (which are FDA approved) by. Ca²⁺ complexation are able to trigger the reversible opening of the tight-junction between the cells and to allow the paracellular transport of peptides (Luessen et al., 1996, Junginger and Verhoef, 1998). Chitosans have been shown to have similar properties to reversibly open the tight junctions. This mechanism is thought to occur by ionic charge transfer between the positive charge of chitosan molecule and the negative charges (sulfate and sialine groups) of the glycocalix (Thanou et al., 2000).

The pulmonary route is also being used for the effective delivery of drugs into the systemic circulation. For a long time, the lung has been used for the administration of drugs for the treatment of local conditions. However, more recently, spurred on by the advent of novel delivery devices, there is a growing interest in the use of the lung for the systemic delivery of challenging molecules, such as peptides and proteins, as well as analgesic agents and even vaccines (Vyas and Khar, 2002). The larger surface area of the lung is well known, although, interestingly, the permeability of the lung tissue in itself is not that different from other mucosal surfaces; it is the large area that provides for the rapid absorption. The challenging aspect still remains unanswered are the mode of delivery for liposomally encapsulated drug to lungs. Metered dose inhalers (MDI) are currently being reformulated as a result of the ban being implemented throughout the world by the United Nations on the use of chlorofluora carbons (CFCs). To meet this challenge, one such alternative is the development of new and improved “Dry Powder Inhaler (DPI)” system that will allow inhalants administration of all drugs presently delivered with MDIs.

Much attention has been paid to the use of the nasal route for the systemic delivery of drugs that are conventionally administered by injection. The nose has many advantages as a potential site for drug delivery; being readily accessible facilitates self-medication, which may improve patient compliance compared to parenteral routes. The nasal mucosa has a relatively large absorptive surface area and is highly vascularized. Furthermore, the blood is drained directly from the nose into the systemic circulation, thus, avoiding first pass metabolism predominantly by the liver.

Further, the intranasal or pulmonary administration provides a useful way of taking a range of systemic drugs, by overcoming the drawbacks occurring because of oral and intravenous administrations. In certain drugs the pharmacokinetics relating to drug absorption and metabolism administered through the nasal and pulmonary routes are more favorable.

U.S. Pat. No. 4,383,993, this patent discloses a method of nasal administration of natural female sex hormones, 17-β-estradiol and/or progesterone which are adapted for nasal administration. The patent does not teach use of synthetic alternatives, such as levonorgestrel, for nasal administration.

U.S. Pat. No. 4,816,258, assigned to Alza Corporation and WO/2001/037770 having Agile Therapeutics, Inc. as applicant both disclose transdermal delivery systems for administering ethinyl estradiol and levonorgestrel. The said patent publications do not disclose or teach administration of the actives nasally.

WO/2010/066883 assigned to Laboratoire Hra-Pharma, France discloses a method of emergency contraception comprising oral administration of 150 μg of levonorgestrel.

In view of above, current inventors have developed a carrier based nasal and pulmonary drug delivery for contraception both in male and female using two different approach i.e., steroidal contraception using a second generation progesterone derivative Levonorgestrel and peptide contraception using a potent gonadotropin releasing hormone agonist, Leuprolide acetate. It is an objective to enhance and maintain effective therapeutic concentrations of the drug for prolonged period of time in development of pharmaceutically rational drug-delivery systems using liposomes and/or mucoadhesives for maximizing the therapeutic index, reducing the dose/ frequency of dosing, and systemic side-effects and, thereby, reducing the cost of therapy.

### SUMMARY OF INVENTION

The main object of invention is to provide novel delivery methods for delivery of emergency contraceptives comprising levonorgestrel alone or in combination with 17α-ethinyl estradiol through nasal route with a view of overcoming above side-effects of oral delivery of emergency contraceptives.

In another aspect, the invention provides a method to enhance and maintain effective therapeutic concentrations of the drug for prolonged period of time in development of pharmaceutically rational drug-delivery systems using liposomes and/or mucoadhesives.

In another aspect, the invention provides a method for emergency contraception whereby, the said formulation in form solution, suspension or carrier based systems selected from microemulsion and liposomes to the person in need of such therapy.

### DETAILED DESCRIPTION OF INVENTION

The invention will now be described in details in connection with certain preferred and optional embodiments so that various aspects thereof may be more fully understood and appreciated.

Accordingly, the current invention describes novel formulations of levonorgestrel for nasal and/or pulmonary delivery. These formulations have been evaluated for their pharmacokinetic efficacy, pharmacodynamic effects and contraceptive efficacy in animals.

In another embodiment, the formulations as described above can comprise ethyl estradiol either alone or in combination with levonorgestrel in 1:1 molar ratio.

Formulations of invention have shown an enhanced brain uptake and up to 100% contraception in animal experimental models. The invention focuses on the alleviation of reported side-effects of oral contraceptives. The pharmacokinetic studies conclusively demonstrate that these routes of administration allow enhanced brain drug deposition thereby altering Luteinizing hormone and Follicle Stimulating Hormone levels favorably to achieve 100% contraception without noticeable side-effects.

Further, compliance of patients who require long-term medication is better due to the simplicity and ease of administration when compared to the parenteral route. Further, the rate of absorption, plasma concentration and pharmacokinetics following nasal and pulmonary administration are often comparable to that obtained by intravenous medication because of rich vasculature and high permeability of nasal and alveolar mucosa. Intranasal and pulmonary administration of the contraceptive drugs avoids first pass metabolism associated with oral administration.

The target for these hormonal drugs used in contraception is Central Nervous System and reproductive tract (endometrium). Further, the olfactory region of the nose demonstrates significant nose to brain transfer thus helping the contraceptive therapy. In selected drugs the pharmacokinetics relating to drug absorption and metabolism through the nose and lungs are altered in a more favorable manner.

It has been shown that the bioavailability of the steroidal drugs progesterone, 17β-estradiol, 17α-ethinylestradiol when given through nasal and pulmonary routes in rats is superior to bioavailability achieved by administration through oral route. Also, studies involving nasal and pulmonary administration of norethisterone show superiority of this route.

In an embodiment, the present invention provides a formulation for nasal or pulmonary delivery of emergency contraceptive comprising levonorgestrel either alone or in combination with 17α-ethinyloestradiol at a defined dose for emergency contraception.

In further embodiment, the invention provides carrier based systems such as microemulsion and liposomes, solution, suspension for nasal administration of levonorgestrel either alone or in combination with 17α-ethinyloestradiol.

In a preferred embodiment, the invention provides a liposomal formulation for levonorgestrel either alone or in combination with 17α-ethinyloestradiol

The invention further provides a process for manufacturing liposomes. The liposomes of present invention can be prepared by methods known in prior art preferably, the liposomes of current invention are prepared by Thin Film Hydration method or Reverse Phase Evaporation method.

The Thin Film Hydration method comprises dissolving phosphatidylcholine, cholesterol,a-tocopherol (equivalent to 1% w/w of PC) and levonorgestrel in Chloroform:Methanol {1:2} mixture in a round bottom flask. The round bottom flask was rotated at 120 rpm under vacuum at 30° C. and dried under nitrogen atmosphere for 20 minutes to form smooth, uniform and dried film. The film thus formed was hydrated by double distilled water under nitrogen atmosphere at glass transition temperature for 45 minutes. Finally liposomes were annealed at glass transition temperature for 2 hour.

The Reverse Phase Evaporation method comprises dissolving phosphatidylcholine, cholesterol and α-tocopherol (equivalent to 1% w/w of PC) and levonorgestrel in Diethyl ether in a glass boiling tube. Double distilled water injected rapidly with force using 23-gauge hypodermic needle attached to 5 ml syringe in the said glass boiling tube. The boiling tube closed with glass stopper; sonicated for 5 minutes in bath sonicator and was attached to rotary flash evaporator to dry the contents at 37° C. under vacuum until gel formation took place. The vacuum was then released and the tube removed from evaporator and was subjected to vigorous mechanical agitation on vortex mixer for 5 minutes till gel collapses. When gel collapsed to fluid, boiling tube was fitted again to rotary flash evaporator for .removal of organic solvent. The cycle of drying and vortexing was repeated twice. The suspension hence formed was treated to remove last traces of organic solvent under high vacuum for 15min to obtain liposomes. Annealing of liposomes was carried out at room temperature for 2 hour after sonication.

The liposomes hence formed by both abovementioned processes were further characterized for shape, size and size distribution, lamellarity, and PDE and for the contents of phosphatidylcholine and cholesterol. Optimization was carried out by selection of various formulations and process variables.

Liposomes hence prepared can be administered nasally in form of dispersion, solution and dry powder form.

Alternatively, the solution as described above can comprise ethyl estradiol either alone or in combination with levonorgestrel.

In another preferred embodiment, the invention provides a chitosan solution for nasal administration of levonorgestrel. The said formulation comprises levonorgestrel liposomes, as prepared above, alongwith pharmaceutically acceptable excipients selected from at least one solubalizer, from the group of propylene glycol, polyethylene glycol 400, ethanol or combinations thereof, at least, one surfactant, preferably, acconon, at least one solvent selected such as chitosan acetate in an aqueous base.

The invention further provides process for manufacturing the chitosan solution for nasal administration of levonorgestrel. The process comprises manufacturing of chitosan solution and levonorgestrel liposome solution separately and mixing the two solutions to form final formulation.

The process for manufacturing chitosan solution comprises dispersing accurately weighed chitosan powder in 0.01% acetic acid. The dispersion was then stirred using magnetic stirrer till the clear solution is obtained. The solution thus formed was kept overnight in freeze to remove entrapped air. The final chitosan concentration was adjusted to 1% w/v.

The process for manufacture of levonorgestrel comprises weighing accurately and dispersing levonorgestrel in double distilled water and sonicating for approximately 1 hr to get particle size in range of 10-15 micron. The suspension was diluted to get final drug concentration of 2 mg/ml. The resulting suspension was further diluted with the equal volume of Chitosan solution, as prepared above. The resulting mixture was mixed well and stored in glass vial in refrigerator till use.

Alternatively, the levonorgestrel, as such, can be incorporated in the prepared solution.

Alternatively, the solution as described above can comprise ethyl estradiol either alone or in combination with levonorgestrel.

In another preferred embodiment, the invention provides dry powder formulation for nasal administration of levonorgestrel. The said dry powder formulation comprises levonorgestrel along with pharmaceutically acceptable excipients selected from carrier such as, sugars and sugar alcohols preferably, selected from the group Sorbolac 400 or Pharmatose 325 M and HPMC.

Alternatively, a mixture of drugs encapsulated in carrier systems such as, but not limited to, liposomes and then freeze-drying it to form a free flowing powder and then mixing with HPMC.

Alternatively, the powder as described above can comprise ethyl estradiol either alone or in combination with levonorgestrel.

In another embodiment the invention provides a method of emergency contraception, wherein the person in need of such treatment comprising nasal administration of levonorgestrel.

In an embodiment the invention provides a method of emergency contraception comprsing administration of levonorgestral in combination with ethyl estradiol in molar ratio of 1:1.

The said formulation is administered in form of solution, suspension or carrier based systems selected from microemulsion and liposomes.

In another embodiment the invention provides use of abovementioned formulation for emergency contraception.

The instant invention is more specifically explained by following examples. However, it should be understood that the scope of the present invention is not limited by the examples in any manner. It will be appreciated by any person skilled in this art that the present invention includes following examples and further can be modified and altered within the technical scope of the present invention.

### EXAMPLE 1

Liposomes:

| Sr. No. | Ingredients | Composition |
|---|---|---|
| 1. | Levonorgestral and/or Ethyl Estradiol | 1:1 or 1 |
| 2. | Drug:HSPC:DSPG:Cholesterol | 1:12:1:1 (Molar Ratio) |
| 3. | α-tocopherol | Equivalent to 1% w/w of |
| | | Phosphatidyl Choline |
| 4. | Chloroform:Methanol (as solvent) | 1:2 |
| 5. | Distilled Water | 1 ml/20 mg of solid |
| | (as Hydration medium) | content |

Process for Manufacture of Liposomes:

Thin Film Hydration:
(a) Dissolving phosphatidylcholine, cholesterol and a-tocopherol (equivalent to 1% w/w of PC) and levonorgestrel and/or Ethyl Estradiol in Chloroform : Methanol {1:2} mixture in a round bottom flask;
(b) drying the solution of step (a) by rotating the round bottom flask at 120 rpm under vacuum at 30° C. and drying under nitrogen atmosphere for 20 minutes till formation of smooth, uniform and dried film;
(c) hydrating film formed in step (b) by double distilled water under nitrogen atmosphere at glass transition temperature for 45 minutes, and
(d) annealing liposomes at glass transition temperature for 2 hour.

Reverse Phase Evaporation Method:
(a) Dissolving phosphatidylcholine, cholesterol and a-tocopherol (equivalent to 1% w/w of PC) and levonorgestrel and Ethyl Estradiol in Diethyl ether in a glass boiling tube;
(b) injecting double distilled water rapidly with force using 23-gauge hypodermic needle attached to 5 ml syringe in the said glass boiling tube;
(c) sonicating the solution of step (b) for 5 minutes in bath sonicator,
(d) drying the solution of step (c) in rotary flash evaporator at 37 ° C. under vacuum until gel formation took place;
(e) vigorously agitating gel of step (d) on vortex mixer for 5 minutes till gel collapses;
(f) repeating steps (d)-(e) twice to form liposomes, and
(g) annealing of liposomes of step (f) at glass transition temperature for 2 hour, after sonication.

### EXAMPLE 2

Solution:

| | | |
|---|---|---|
| Sr. No. | Ingredients | Composition |

| | | |
|---|---|---|
| 1. | Levonorgestrel and/or Ethyl Estradiol | 0.5-1% |
| 2. | Propylene glycol | 5-10% |
| 3. | Polyethylene glycol 400 | 10-15% |
| 4. | Ethanol | up to 5% |
| 5. | Acconon | up to 1% |
| 6. | Chitosan Acetate | 0.5-1% |
| 7. | Distilled Water | up to 100% |

Process for Manufacture:
(a) Dispersing accurately weighed chitosan powder in 0.01% acetic acid;
(b) stirring dispersion of step (a) using magnetic stirrer till the clear solution is obtained;
(c) keeping the solution of step (b) overnight in freeze to remove entrapped air;
(d) adjusting final chitosan concentration to 1% w/v;
(e) accurately weighing and dispersing levonorgestrel and/or ethyl estradiol in double distilled water or alternately, liposomes of levonorgestrel and/or ethyl estradiol of example 1;
(f) sonicating solution of step (e) for approximately 1 hr to get particle size in range of 10-15 micron to form a suspension;
(g) diluting suspension of step (f) to get final drug concentration of 2 mg/ml, and
(h) further diluting suspension of step (g) was with the equal volume of Chitosan solution.
(i) Accurately weighing Propylene glycol, Polyethylene glycol 400 and Ethanol;
(j) stirring mixture of step (i) using magnetic stirrer till the clear solution is obtained, and
(k) adding solution of step (h) under constant stirring using magnetic stirrer.

### EXAMPLE 3

Powder:

| Sr. No. | Ingredients | Composition |
|---|---|---|
| 1. | Micronized drug/drug encapsulated in liposome | 0.5-1% |
| | (example 1) | |
| 2. | HPMC | 1% |
| 3. | Sorbolac 400 or Pharmatose 325 M | up to 100% |

Process for Manufacturing:
(a) Centrifuging optimized liposomal batches prepared according to Example 1, to form pellets;
(b) Hydrating pellet of step (a) with the required quantity of hydration medium containing sugar to obtain different lipid: sugar mass ratio to form a suspension;
(c) freezing suspension of step (b) at −40° C. overnight and drying under negative displacement pressure for 24 h to obtain a porous cake;
(d) mixing porous cake of step (c) either with Sorbolac 400(lactose) or Pharmatose 325 M(trehalose) and HPMC to form powder;
(e) sieving powder of step (d) through #200 and #240 sieves
(f) filling Capsules (size ‘2’) with individually weighed powder (10 mg) of step (e) containing 250 μg drug and packed under nitrogen atmosphere in high-density polyethylene (HDPE) bottles containing silica bags as desiccant.

### EXAMPLE 4

Microemulsion:

| Sr. No. | Ingredient | % w/w |
|---|---|---|
| 1 | Levonorgestrel and/or Ethylestradiol | 0.5-1 |
| 2 | Capmul MCM | 4.2 |
| 3 | Acconon CC6 | 30.4 |
| 4 | Ethanol | 7.6 |
| 5 | Distilled Water | 57.3 |
| 6 | Polycarbophil/Chitosan | 0.5 |

Process for Manufacture:
a) Accurately weighing Capmul MCM, Acconon CC6 and Ethanol;
b) stirring mixture of step (a) using magnetic stirrer till the clear solution is obtained;
c) accurately weighing and dispersing Levonorgestrel and/or Ethylestradiol in the mixture of step (b), and
d) adding double distilled water containing polycarbophil/chitosan to mixture of step (c) under constant stirring using magnetic stirrer.

### EXAMPLE 5

### Suspension

Levonorgetral and/or Ethyl Estradiol in micronized form in concentration of 0.5 - 1% is suspended in 1% chitosan acetate solution in water.

### EXAMPLE 6

Pharmacodynamic Studies:

Study Methodology

Size of the drug particles and liposomes in all the formulations was kept between 10-15 μm, as the particles with 10-20 μm are all deposited in the nasal cavity, whereas particles smaller than 1 μm pass with inspired air into the lungs.

Suspension containing 10-μg levonorgestrel was administered intranasally in three different group of rats. Similarly, one group of animals were treated with 10-μg of levonorgestrel suspension administered orally.

Blood samples were collected at specific time points and plasma levonorgestrel concentrations were estimated. The drug plasma concentration at each sampling time point were plotted against time in hr.

Observations

Various pharmacokinetic parameters (Cₘₐₓ, Tₘₐₓ and t_{1/2}) were determined from drug plasma concentration-time curve Plasma levels of levonorgestrel after a single oral dose indicate that a considerably higher level of levonorgestrel (Cₘₐₓ 14.4 ng/mL) occurs with Tₘₐₓ of 2.1 h as compared to levonorgestrel suspension, levonorgestrel formulations given intranasally (Cₘₐₓ 7.13 ng/mL, 6.1 ng/mL, 5.24 ng/mL) at Tₘₐₓ of 4.2 h, 4.6 h and 4.6 h respectively. Levels of levonorgestrel fall precipitously to levels-below 1 ng/mL in all the cases. levonorgestrel suspension, evonorgestrel powder and liposomal levonorgestrel formulations were having significantly less bioavailability (25-32%). The mucociliary clearance under normal conditions rapidly clears the applied material since there is a little time of contact between the drug and the mucosa. When the drug was formulated with mucoadhesive agents, CS (LN+CS) and CP (LN+CP), significant improvement in F* of drug were observed (101.70% and 99.42% respectively). Plasma half-lives (t_{1/2}) were also significantly increased from 7.0 hr to 55.7 hr and 52.9 hr, having the T. of 4.4 hr and 5.0 hr and Cₘₐₓ of 4.73 ng/mL and 4.70 ng/mL respectively for LN+CS and LN+CP formulations. Prolonging the contact time of the drug with the absorptive surfaces by means of appropriate mucoadhesive agent contributed to increase in the F* of intranasally administered drug. The clearance of administered drug was delayed by using mucoadhesive polymers such as CS and CP. CS acts by opening tight junction between epithelial cells. It may also enhance the absorption of drugs by being a useful biciadhesive and slowing mucociliary transport. Carbopol hydrogel is a thin liquid at acidic pH but it gels at physiological pH and thus has great potential for nasal delivery of drugs. When the t_{1/2} value of orally administered formulation was compared to nasally administered mucoadhesive formulations, significant increases in t_{1/2} were observed (16.9 h to 52.9 h-55.7 h).

### CONCLUSION

The results clearly indicate that the dosing interval can be changed to once in two days from daily oral administration without changing the dose. The reduction in the drug dose and maintenance of therapeutic concentration in blood plasma of the drug for at least up to the 48 h is expected to reduce the reported side effects in humans and probably the cost of the therapy due to lower dose.

### EXAMPLE 7

Pharmacodynamic Studies:

Methodology:

The animals were administered with different formulations intranasally for four weeks and allowed for mating during the treatment period.

Observations:

Animals when treated nasally with levonorgestrel failed to show contraceptive efficacy, may be due to short plasma half-lives of the drug. However, in case of levonorgestrel with chitosan and levonorgestrel with carbolpol cent percent anti-fertility was observed even formulations were administered on alternate days. These results are in agreement with pharmacokinetics, which further confirms the contraceptive efficacy of proposed formulations for prolonged period of time.

### EXAMPLE 8

Study Methodology

levonorgestrel suspension, plain levonorgestrel and liposomal levonorgestrel Dry powder formulations containing 10-μg LN were administered intratracheally in three different group of rats. Similarly, 10-μg of LN suspension was administered orally. Blood samples were collected at specific time points and plasma LN concentrations were estimated. The drug plasma concentration at each sampling time points were plotted against time in hr.

Observations

Various pharmacokinetic parameters (Cₘₐₓ, Tₘₐₓ and t_{1/2}) were determined from drug plasma concentration-time curve. The area under the plasma level curve was calculated by the trapezoidal rule. The AUC following oral and intratracheal administration of formulations were found to be significantly different (p<0.05). However, no significant difference (p>0.05) was observed in AUC after intratracheal administration of these formulations. The F* values after intratracheal administration were 97.6%, 109.88%, and 98.55% for levonorgestrel suspension, plain levonorgestrel and liposomal levonorgestrel-Dry powder formulations, .respectively. Following oral drug delivery, Cmax of 14.4 ng/mL was followed by decline in plasma concentration with t1/2 of 16.9 hrs. In contrast, pulmonary delivery gave effective plasma drug concentration for the period of 56 to 60 hrs with the zero-order release kinetics following Cmax of 4.40, 4.42, and 4.20 ng/mL for levonorgestrel suspension, plain levonorgestrel and liposomal levonorgestrel-Dry powder formulations, respectively.

### CONCLUSION

Pulmonary delivery of all 3 formulations resulted in similar pharmacokinetic behavior because of the similarity in lipophilicity and size of the drug and liposomes. Slow and prolonged absorption of the drug after pulmonary delivery significantly reduces Cₘₐₓ and is also expected to reduce dose-dependent progestronic side effects associated with orally administered levonorgestrel.

1. An emergency contraceptive formulation for nasal and/or pulmonary administration comprising of levonorgestrel and mucoadhesive agent.

2. The emergency contraceptive formulation as claimed in claim 1, wherein the mucoadhesive agent is selected from Polycarbophil or Chitosan.

3. The emergency contraceptive formulation as claimed in claim 1, wherein the formulation is in form of solution, suspension, powder or carrier based systems selected from microemulsion and liposomes.

4. The emergency contraceptive formulation as claimed in claim 3, wherein solution comprises Levonorgestral, at least one solubilizer selected from propylene glycol, polyethylene glycol 400, ethanol or combinations, at least, one surfactant, such as, acconon, at least one solvent selected such as chitosan acetate in a aqueous base.

5. The emergency contraceptive formulation as claimed in claim 3, wherein suspension comprises Levonorgestral in a carrier solution comprising 1% Chitosan acetate as carrier.

6. The emergency contraceptive formulation as claimed in claim 3, wherein microemulsion comprises levonorgestrel at least one surfactant such as, Capmul MCM, Acconon CC6; Ethanol; water and mucoadhesive agents such as Polycarbophil / Chitosan.

7. The emergency contraceptive formulation as claimed in claim 3, wherein dry powder formulation comprises levonorgestrel alongwith pharmaceutically acceptable excipients selected from carrier such as, sugars and sugar alcohols preferably, selected from the group of Sorbolac 400 or Pharmatose 325 M and HPMC.

8 The emergency contraceptive formulation as claimed in claim 3, wherein the liposomes comprise lipids such as phosphatidylcholine, cholesterol and a-tocopherol (equivalent to 1% w/w of PC) mixture.

9. The emergency contraceptive formulation as claimed in claim 1, wherein the formulations further comprise ethylestradiol.

10. The emergency contraceptive formulation as claimed in claim 9, wherein Levonorgestral and ethyl estradiol are present in molar ratio of 1:1 .

11. A method of emergency contraception in a human, comprising nasal and/or pulmonary administration of levonorgestrel to said subject.

12. The method as claimed in claim 11, wherein the method further comprises administration of ethylestradiol.

13. The emergency contraceptive formulation as claimed in claim 12, wherein Levonorgestral and ethyl estradiol are administered in molar ratio of 1:1.

14. The method as claimed in claims 11, wherein the levonorgestrel optionally in combination with ethylestradiol is administered in form of solution, suspension, powder or carrier based systems selected from microemulsion and liposomes.

15. Use of medicament comprising levonorgestrel, optionally in combination with ethylestradiol nasally and/or pulmonarily administrable form for emergency contraception.
